(19) United States
(12) Patent Application Publication
(10) Pub. No.: US 2017/0196980 A1
(43) Pub. Date: Jul. 13, 2017

(54) TRANSDERMAL ENHANCER

(71) Applicant: Tsung-Min HSU, Taipei City (TW)

(72) Inventor: Tsung-Min HSU, Taipei City (TW)

(21) Appl. No.: 15/440,613

(22) Filed: Feb. 23, 2017

Related U.S. Application Data
(63) Continuation-in-part of application No. 14/552,568, filed on Nov. 25, 2014, now abandoned.
(60) Provisional application No. 61/911,557, filed on Dec. 4, 2013.

(51) Int. Cl. A61K 47/18; A61K 31/593; A61K 31/714; A61K 9/06; A61K 9/00; A61K 9/107; A61K 9/70; A61K 47/02; A61K 31/4439

(52) U.S. Cl. CPC A61K 47/186; A61K 47/02; A61K 31/593; A61K 31/714; A61K 31/4439; A61K 9/0014; A61K 9/107; A61K 9/7038; A61K 9/06

(57) ABSTRACT

The present invention is related to a composition for enhancing topical and transdermal absorption comprising an alkali material having a pH in the range of about 9.0 to 14.0 and a water-soluble substance in an amount of at least 0.5 wt % more than an amount of the alkali material based on a total weight of the composition. The present invention is also related to a pharmaceutical composition comprising the composition, at least one therapeutically effective amount of a drug or an effective cosmetic ingredient, and a pharmaceutically acceptable carrier. The present invention is also related to a method for enhancing and delivering a drug through a body surface to minimize or eliminate the possibility of skin damage, irritation or sensitization while the above-described composition or the pharmaceutical composition is crossing patient's skin or mucosal tissue.

The present invention is a continuation-in-part application that claims the benefit of U.S. patent application Ser. No. 14/552,568 filed on Nov. 25, 2014.

### BACKGROUND OF THE INVENTION

### 1. Field of the Invention

The present invention relates to topical and transdermal administration of pharmacologically active agents and more particularly relates to a composition that enhances the permeability of skin or mucosal tissue to topically apply pharmacologically active agents.

### 2. Description of the Prior Arts

The deliveries of drugs through oral treatment or injection are inconvenient. For example, regarding oral treatment, some medicine may not endure the strong acid in stomach, or may even result in serious gastrointestinal side effects. Moreover, when some medicines are injected topically, irritation will occur. Thus, transdermal administration, a means of delivery of drugs through the skin, provides many advantages such as comfort, convenience and non-invasiveness.

However, skin is a structurally complex and thick membrane. While drugs or molecules are moving into deep skin, firstly, they must penetrate the stratum corneum, the epidermis, the dermis, and the capillary walls into the blood stream or lymph channels. To be absorbed, drugs or molecules must overcome different degrees of resistance for penetration into each type of cell layer. For instance, the stratum corneum is a thin layer of dense highly keratinized cells approximately 10-15 microns thick over most of the body. It is believed that the high degree of keratinization within these cells as well as their dense packing creates in most cases a substantially impermeable barrier for absorption.

Even though the prior arts have disclosed the water-soluble substance with the pH over 8.5 that helps the water-soluble substance get into the skin, they neither teach that the alkali material with the pH over 8.5 would cause irritation and corrosion of the stratum corneum layer of the skin, nor teach how to prevent and alleviate the irritation and corrosion caused by the alkali material with the pH over 9.

### SUMMARY OF THE INVENTION

To enhance the penetration of chemical materials, natural extract or organic polypeptides via the skin or the mucosa tissue of oral cavity, anus, and reproductive organs to body for treatment, health care or cosmetic purposes, the objective of the present invention is to provide a composition for enhancing topical and transdermal absorption.

To achieve the above objective, the composition comprises an alkali material having a pH in the range of about 9.0 to 14.0 and a water-soluble substance in an amount of at least 0.5 wt % more than an amount of the alkali material based on a total weight of the composition.

The term “alkali material” as used herein refers to an agent that releases free hydroxide ions in an aqueous environment. Accordingly, any alkali material that has a pH value over 9.0 can open a channel between a protein portion (e.g. keratin) and a lipid bilayer at a stratum corneum layer of skin to permit water-soluble material with molecular weight less than 2,000 from penetrating into the body.

In one embodiment, example of the alkali material is selected from the group consisting of an organic base and an inorganic base, wherein the organic base includes a nitrogenous base. Preferably, the organic base includes, but is not limited to, trimethylamine. triethylamine, methylamine, ethylamine, dimethylamine, diethylamine, alkylamine, alkyldiamine, alkyltriamine, aromatic amine, tetramethylammonium hydroxide, tetraethylammonium hydroxide, tetrapropyl-ammonium hydroxide, tetramethylammonium bisulfide, tetraethylammonium bisulfide, pyridine and derivatives of pyridine, imidazole and derivatives of imidazole, benzimidazole and derivatives of benzimidazole, histidine and derivatives of histidine, phosphazene base, pydroxides of some organic cations, and combinations of two or more thereof. Wherein the aromatic amine includes, but is not limited to, aniline, toluidine, anisidine, and N-methylaniline. The inorganic base includes, but is not limited to, ammonium hydroxide, sodium hydroxide, potassium hydroxide lithium hydroxide, calcium hydroxide, magnesium hydroxide, cesium hydroxide, lithium carbonate, potassium carbonate, potassium phosphate, sodium carbonate, sodium phosphate, cesium carbonate and combinations thereof.

The term “water-soluble substance” as used herein refers to an agent that forms hydrogen bonding in an aqueous environment.

In another embodiment, example of the water-soluble substance includes, but is not limited to, compound posing amine functional group: primary amine, secondary amine, tertiary amine, and quaternary ammonium cations; compound posing carboxylic acid functional group: fatty acid, amino acid, keto acid, aromatic carboxylic acid, dicarboxylic acid, tricarboxylic acid, and alpha hydroxyl acid; compound posing sulfhydryl functional group: aliphatic and aromatic sulfhydryl compound; compound posing hydroxyl functional group: aliphatic and aromatic alcohol, glycols, triols, and polyals; compound posing phosphoric acid functional group: potassium phosphoric acid, sodium phosphoric acid, and benzylphosphoric acid. Preferably, the water-soluble substance is gelatin, agar, alginic acid, sodium alginate, dextrin, xanthan gum, gum arabic powder, hydroxyethylcellulose, hydroxypropylcellulose, hydroxypropylmethylcellulose, polyvinylpyrrolidone, partially saponified polyvinyl alcohol, methylcellulose, pullulan, partially pregelatinized starch, or saccharides. These substances may be used individually or in combination of two or more.

However, alkali material with the pH over 9.0 may hurt the skin, inasmuch as alkali material would release free hydroxide ions to result in irritation and corrosion due to the pH over 9. Therefore, the water-soluble substance of concentration of at least 0.5 wt % more than the amount of the alkali material based on a total weight of the composition forms hydrogen bonding in the aqueous environment to decrease the activity of water and the degree of dissociation of the alkali material, and to reduce the activity of free hydroxide ion and avoid irritation and corrosion.

On the other hand, by means of composition having the water-soluble substance of at least 0.5 wt % more than the amount of the alkali material and alkali material with the pH over 9.0, wherein the alkali material can constantly release free hydroxide ions to open a channel between the protein portion (e.g. keratin) and lipid bilayer at the stratum corneum layer of skin, and then the channel can constantly open to permit water-soluble material with molecular weight less than 2,000 through the body.

The advantage of the composition in accordance with the present invention not only clearly provides a mechanism of the alkali material affecting the region of the skin and the water-soluble substance penetration, but also provides a means to reduce harm caused by the alkali material. In brief, the alkali material with the pH over 9.0 of the composition can release free hydroxide ions in an aqueous environment, and the water-soluble substance of at least 0.5 wt % more than the amount of the alkali material based on the total weight of the composition can form hydrogen bonding in an aqueous environment to enhance the penetration of substances such as drugs or molecules and to avoid irritation and corrosion of the skin. As for a water-insoluble substance, it could be dissolved in water with the addition of a solution such as alcohol to penetrate into the skin.

In a related aspect of the present invention, a pharmaceutical composition of matter is provided for delivering a drug through a body surface using the composition comprising alkali material with the pH over 9.0 and water-soluble substance with at least 0.5 wt % more than the amount of the alkali material based on the total weight of the composition as a permeation enhancer. Generally, the formulation comprises (a) at least one therapeutically effective amount of a drug or an effective cosmetic ingredient, (b) alkali material with the pH over 9.0 and water-soluble substance in an amount effective to enhance the flux of the drug through the body surface without causing damage thereto, and (c) a pharmaceutically acceptable carrier suitable for topical or transdermal drug administration. The composition may be in any form suitable for application to the body surface, and may comprise, for example, a cream, lotion, solution, gel, ointment, paste or the like, and/or may be prepared so as to contain liposome, micelles, and/or microspheres. The composition may be directly applied to the body surface or may involve use of a drug delivery device. In one case, it is preferred although not essential that water be present in order for the water-soluble substance to form hydrogen bonding and thus decrease the activity of water and the degree of dissociation of the alkali material, to reduce the activity of free hydroxide ion and avoid irritation and corrosion. In another case, a formulation or drug reservoir may be aqueous, i.e., contain water, or may be nonaqueous and used in combination with an occlusive overlayer so that moisture evaporating from the body surface is maintained within the formulation or transdermal system during administration.

In another aspect of the present invention, a method is provided for minimizing or eliminating the possibility of skin damage, irritation or sensitization while the above-described composition is crossing patient's skin or mucosal tissue.

### BRIEF DESCRIPTION OF THE DRAWINGS

FIG. 1 shows effects of cream on treated skin indicated by Corneometer CM 825 within 7 days; wherein A is a cream from example 1 of the present invention, B is a commercially available cream of OLAY®, C is a commercially available cream of Clinique®, and D is the commercially available vaseline.

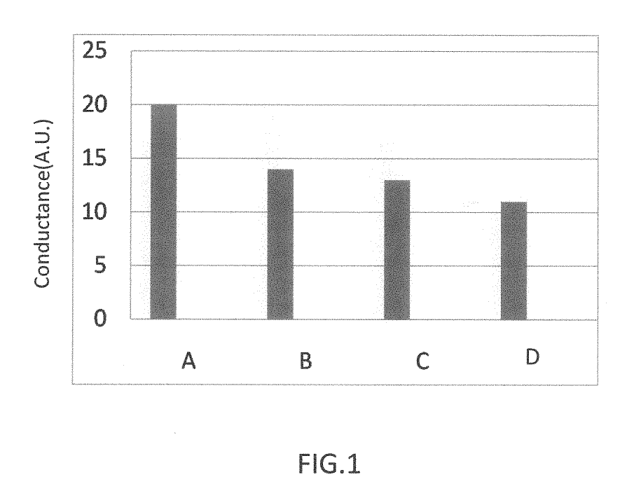

FIG. 2 shows effects of cream on treated skin indicated by transepidermal water loss (TEWL) within 7 days; wherein A is the cream from example 1 of the present invention, B is the commercially available cream of OLAY®, C is the commercially available cream of Clinique®, and D is the commercially available vaseline.

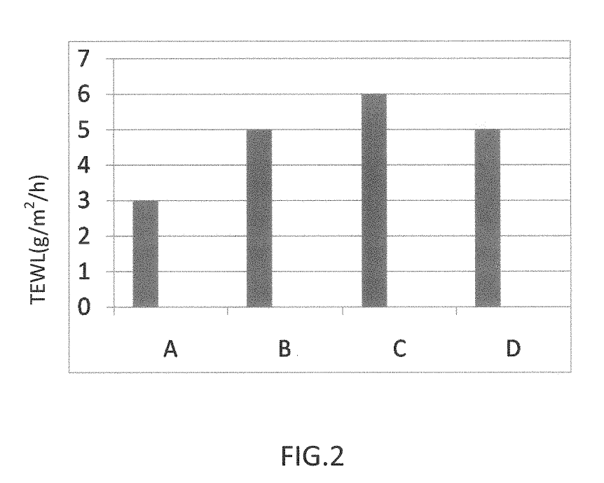

FIG. 3 is a measurement of D-Squame Scores on treated skin indicated within 7 days; and, wherein A is the cream from example 1 of the present invention, B is the commercially available cream of OLAY®, C is the commercially available cream of Clinique®, and D is the commercially available vaseline.

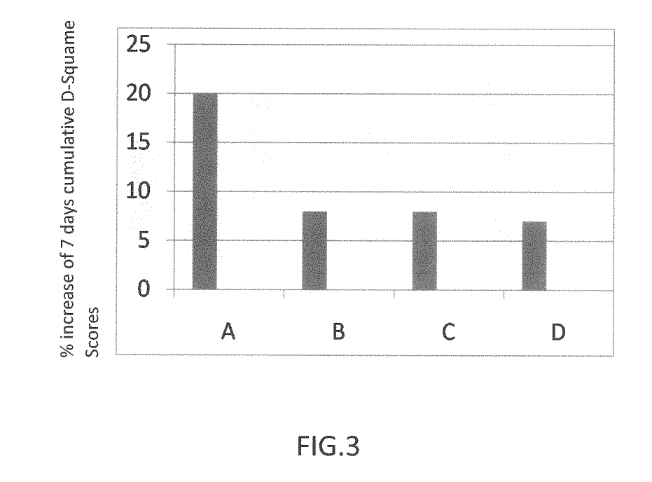

FIG. 4 shows the cumulative score of skin smoothness of 16 patients within 14 days, wherein A is the cream from example 1 of the present invention, B is the commercially available cream of OLAY®, C is the commercially available cream of Clinique®, D is the commercially available vaseline.

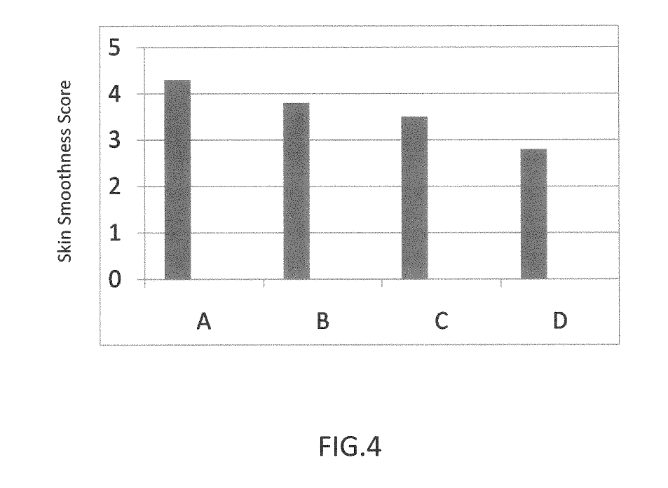

### DETAILED DESCRIPTION OF THE PREFERRED EMBODIMENTS

Other objectives, advantages and novel features of the invention will become more apparent from the following detailed description.

### EXAMPLE 1

Cream, as also well known in the art, is viscous liquids or semisolid emulsions, either oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. An in vitro skin permeation study was conducted using an oil-in-water cream which has pH value at 12.0 as shown in the following Table 1.

TABLE 1
Weight percent of each component (based on total
solution weight)

| | |
|---|---|
| Distilled water | 72.8% |
| Ornithine HCl | 2.5% |
| Arginine | 2.7% |
| PCA-Na (sodium pyrrolidone carboxylate) | 2.3% |
| Lactic acid | 3.0% |
| Glycerin | 4.0% |
| NaOH (1:2 diluted with water) | 6.3% |
| Cetyl alcohol | 3.4% |
| Cetrimonium chloride | 2.0% |
| Brij 720 | 0.8% |
| Brij 30 | 0.2% |

### EXAMPLE 2

Generally, the underlying surface of the transdermal device, i.e., the skin contact area, has an area in the range of about 5 cm² to 200 cm², preferably 5 cm² to 100 cm², and more preferably 20 cm² to 60 cm². The area will vary with the amount of drug to be delivered and the flux of the drug through the body surface. Larger patches will be necessary to accommodate larger quantities of drug, while smaller patches can be used for smaller quantities of drug and/or drugs that exhibit a relatively high permeation rate.

An in-vitro skin permeation study was conducted using two vitamin D3 transdermal systems. The formulations used to prepare these systems are listed as shown in the following Table 2, which includes weight percent of each ingredient in the formulation.

TABLE 2
Weight percent of each component based on total
weight of each patch

| Vitamin D3 patch A | | Vitamin D3 patch B | |
|---|---|---|---|
| Vitamin D3 | 1.97% | Vitamin D3 | 1.97% |
| Diethylene | 14.80% | Diethylene | 9.87% |
| glycolmonoethylether | | glycolmonoethylether | |
| Polyvinyl pyrrolidone | 11.85% | Polyvinyl pyrrolidone | 6.90% |
| Polyisobutylene adhesive | 68.82% | Polyisobutylene adhesive | 56.26% |
| (Duro-tak ® 87-6908) | | (Duro-tak ® 87-6908) | |
| NaOH (1:2 diluted with | 1.58% | NaOH (1:2 diluted with | 3.00% |
| water) | | water) | |
| Sodium lauroylsarcosine | 0.99% | Lysine | 8.00% |
| | | Arginine | 8.00% |
| | | cetrimonium chloride | 3.00% |
| | | Brij 30 | 3.00% |

All ingredients were mixed together with Duro-tak®, Polyisobutylene adhesive, and laminated 50 mil.wet on a silicon coated releasing liner and cut into round discs. This 50 mil. thick patch was dried at 60° C. oven to remove n-heptane from the adhesive. The dried patch was then laminated with a polyethylene backing to form a vitamin D3 in matrix A patch having pH value at 11.0 and a vitamin D3 in matrix B patch having pH value at 13.5.

The in-vitro permeation of vitamin D3 through human cadaver skin from these discs was performed using Franz-type diffusion cells with a diffusion area of 1 cm². The volume of receiver solution was 8 ml. Human cadaver skin was cut to desired size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution. Twelve diffusion cells were used for each formulation. The measured skin permeations at the skin/patch interface were listed in Table 3.

TABLE 3
Skin permeation of vitamin D3 patch A and patch B

| | |
|---|---|
| vitamin D3 patch A | 1.9 (μg/cm²/24 hrs) |
| vitamin D3 patch B | 5.2 (μg/cm²/24 hrs) |

The cumulative amount of vitamin D3 of the patch A permeated across human cadaver skin at 24 hours is 1.9 μg/cm²/24 hrs; the cumulative amount of vitamin D3 of the patch A permeated across human cadaver skin at 24 hours is 5.2 μg/cm²/24 hrs.

### EXAMPLE 3

Gels are semisolid, as will be appreciated by those working in the field of pharmaceutical formulation suspension-type systems. Single-phase gels contain organic macromolecules distributed substantially uniformly throughout the carrier liquid, which is typically aqueous, but also, preferably, contains an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

An in vitro skin permeation study was conducted using two vitamin B12 transdermal gels. The formulations used to prepare these gels are listed in Table 4, which includes weight of each component in the formulations.

TABLE 4
Weight of each component based on total weight of
each transdermal gel

| | |
|---|---|
| Vitamin B12 gel A* | Vitamin B12 gel B |

Part I gel

| | | | | | |
|---|---|---|---|---|---|
| Methylcobalamin | 0.5 | mg | methylcobalamin | 0.5 | mg |
| hyaluronic acid | 0.5 | g | hyaluronic acid | 0.5 | g |
| Lysine | 1.0 | g | lysine | 1.0 | g |
| distilled water | 98 | g | distilled water | 98 | g |

Part II gel

| | | |
|---|---|---|
| NaOH (1:2 diluted | 10.0 | g |
| with water) | | |
| hyaluronic acid | 0.5 | g |
| distilled water | 74.5 | g |
| Lysine | 15.0 | g |

*Mix Part I gel and Part II gel together before application on the skin.

The measured skin permeations at the skin/gel interface were listed in Table 5.

TABLE 5
Skin permeation of vitamin B12 gel A and gel B

| | |
|---|---|
| Vitamin B12 gel A | 0.09 (μg/cm²/24 hrs) |
| Vitamin B12 gel B | 0 (μg/cm²/24 hrs) |

The cumulative amount of vitamin B12 of the gel A permeated across human cadaver skin at 24 hours is 0.09 μg/cm²/24 hrs; the cumulative amount of vitamin B12 of the gel B permeated across human cadaver skin at 24 hours is 0 μg/cm²/24 hrs.

### EXAMPLE 4

In the instant example, betaine is employed as the water-soluble substance. Betaine functions as an osmolyte to maintain the stasis of osmosis of a cell. The composition in accordance of the instant invention as demonstrated in the current example that comprises betaine contributes in maintaining the functions of enzymes within cells, and also helps protecting the structures of proteins within the cells from degradation. The integrity of the cell membrane of the cell is therefore benefited from using betaine as a component of the composition.

TABLE 6
Formulations

| | Weight | | |
|---|---|---|---|
| | (wt %) | | |
| | Sample A | Sample B | Sample C |
| Drug (Omeprazole) | 3.0 gm | 3.0 gm | 3.0 gm |
| | (16.57%) | (15.30%) | (14.22%) |
| Distilled water | 2.0 gm | 2.0 gm | 2.0 gm |
| | (11.05%) | (10.20%) | (9.48%) |
| Dimethyl sulfoxide | 2.0 gm | 2.0 gm | 2.0 gm |
| | (11.05%) | (10.20%) | (9.48%) |
| Benzyl alcohol | 0.4 gm | 0.4 gm | 0.4 gm |
| | (2.20%) | (2.04%) | (1.89%) |
| NaOH | 0.45 gm | 0.45 gm | 0.45 gm |
| | (2.48%) | (2.30%) | (2.13%) |
| Betaine | 0 | 0.5 gm | 1.0 gm |
| | | (2.55%) | (4.74%) |
| N-methyl glucamine | 0 | 1.0 gm | 2.0 gm |
| | | (5.10%) | (9.48%) |
| Polyisobutylene adhesive | 8.0 gm | 8.0 gm | 8.0 gm |
| | (44.20%) | (44.82%) | (37.91%) |
| Hydrocarbon resins | 1.5 gm | 1.5 gm | 1.5 gm |
| | (8.28%) | (7.65%) | (7.11%) |
| Mineral oil | 0.25 gm | 0.25 gm | 0.25 gm |
| | (1.38%) | (1.28%) | (1.18%) |
| polyvinylpolypyrrolidone | 0.5 gm | 0.5 gm | 0.5 gm |
| | (2.76%) | (2.55%) | (2.37%) |

More importantly, betaine, posing hydroxyl functional group, as employed in the instant example, disturbs the framework formed by hydrogen bonds of pure water, when the composition in accordance with the instant invention is dissolved therein. The solute, betaine, therefore decreases the dissociation rate of a co-dissolved base, so as to alleviate the stimulating and irritating nature of the rapidly dissociating base. As a result, the composition in accordance with the instant invention as exemplified in the example is useful and effective in mitigating the extremely unpleasant stimuli brought by the rapid dissociation of the base during the administration of a drug whose transdermal delivery is enhanced by the base. It is preferred that the amount of the betaine posing hydroxyl functional group may range from 2 wt % to 35 wt % based on a total weight of the composition.

In the instant example, the water soluble substance consists of Betaine and N-methyl glucamine. The water-soluble substance is of an amount of at least 0.5 wt % more than the alkali material, which is NaOH in the instant example. It has been demonstrated that Sample A that comprises an alkali material of an amount of 2.48 wt % based on the total weight of the composition, and a totality of water-soluble substance of the amount of 0 wt %. In contrast, Sample B comprises a totality of water-soluble substance of an amount of 7.65 wt %, which is 5.35 wt % more than the amount, 2.30 wt %, of the alkali material; Sample C comprises a totality of water-soluble substance of an amount of 14.22 wt %, which is 12.09 wt % more than the amount, 2.13 wt %, of the alkali material.

TABLE 7
Transdermal absorption study (pig skin)

| | Permeation rate | | |
|---|---|---|---|
| | (ug/cm²) | | |
| | Sample A | Sample B | Sample C |
| 5 hours | 42.5 | 33.4 | 25.2 |
| 10 hours | 156.2 | 101.7 | 63.7 |
| 24 hours | 539.3 | 402.9 | 236.6 |
| 48 hours | 1103.6 | 885.5 | 506.2 |
| 72 hours | 1542.7 | 1205 | 682.6 |

The severities of skin irritation due to the application of Samples A-C are quantified with Draize method as listed in the following table.

TABLE 8
Animal skin irritation study (Draize method: white New
Zealand rabbit)

| | Sample A | Sample B | Sample C |
|---|---|---|---|
| primary irritation index | 5.2 | 1.1 | 0.5 |

The severity of irritation indicated by the primary irritation index is as listed in the following Table 9.

TABLE 9
Evaluation of single dermal irritation

| primary irritation index | Irritation interpretation |
|---|---|
| 0-0.4 | Non-irritant |
| 0.5-1.9 | Slightly irritant |
| 2.0-4.9 | Moderately irritant |
| 5.0-8.0 | Severely irritant |

Foregoing experiments with the above listed Samples A-C in Tables 6-9 have shown that the exampling Samples B and C of the composition in accordance with the invention have significantly lowered the severity of irritation caused in vivo during transdermal administration of Omeprazole.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A composition comprising:
an alkali material having a pH in the range of 9.0 to 14.0; and,
a water-soluble substance in an amount of at least 0.5 wt % more than an amount of the alkali material based on a total weight of the composition;
wherein the alkali material is an inorganic base, the water-soluble substance comprises a betaine posing hydroxyl functional group, and the betaine is in an amount of 2 wt % to 35 wt % based on a total weight of the composition.

2. The composition of claim 1, wherein the inorganic base is selected from the group consisting of ammonium hydroxide, sodium hydroxide, potassium hydroxide lithium hydroxide, calcium hydroxide, magnesium hydroxide, cesium hydroxide, lithium carbonate, potassium carbonate, potassium phosphate, sodium carbonate, sodium phosphate, cesium carbonate and combinations thereof.

3. The composition of claim 2, wherein the inorganic base is sodium hydroxide.

4. The composition of claim 1, wherein the compound posing hydroxyl functional group is selected from the group consisting of aliphatic and aromatic alcohol, glycols, triols, and polyols.

5. A pharmaceutical composition, comprising:
(a) at least one therapeutically effective amount of a drug or an effective cosmetic ingredient;
(b) the composition as claimed in claim 1; and,
(c) a pharmaceutically acceptable carrier suitable for topical or transdermal drug administration.

6. The pharmaceutical composition of claim 5, further comprising liposome, micelles or microspheres.

7. The pharmaceutical composition of claim 6, wherein formulation of the pharmaceutical composition is cream, lotion, solution, gel, ointment or paste.
